(19) United States
(12) Patent Application Publication
(10) Pub. No.: US 2008/0071292 A1
(43) Pub. Date: Mar. 20, 2008

(54) SYSTEM AND METHOD FOR DISPLAYING THE TRAJECTORY OF AN INSTRUMENT AND THE POSITION OF A BODY WITHIN A VOLUME

(76) Inventor: Collin A. Rich, Ypsilanti, MI (US)

Correspondence Address: SCHOX PLC, 209 N. MAIN STREET #200, ANN ARBOR, MI, 48104, US

(21) Appl. No.: 11/858,796

(22) Filed: Sep. 20, 2007

Related U.S. Application Data
(60) Provisional application No. 60/826,340, filed on Sep. 20, 2006.

(51) Int. Cl. A61B 19/00

(52) U.S. Cl. 606/130

(57) ABSTRACT

The present invention includes a system and a method of displaying the trajectory of an instrument and the position of a body within a volume. The preferred method includes the steps of creating a representation of at least a portion of the volume, calculating a trajectory of the instrument relative to a position of the body, and displaying the trajectory of the instrument and the position of the body. The preferred system includes an instrument, an ultrasonic device, a processor coupled to the ultrasonic device, and a display adapted to display the trajectory of the instrument and the position of the body.

### CROSS-REFERENCE TO RELATED APPLICATIONS

This application claims the benefit of U.S. Provisional Application No. 60/826,340 filed 20 Sep. 2006 and entitled “System And Method For Displaying the Trajectory of an Instrument and the Position of a Body within a Volume”, which is incorporated in its entirety by this reference.

### TECHNICAL FIELD

The present invention relates generally to the field of imaging, and more particularly to the field of medical imaging for interventional procedures.

### BACKGROUND

It is common in medical practice to use an imaging device to guide the insertion and/or use of medical devices. For example, ultrasound devices are commonly used to guide the insertion of a biopsy needle. The image generated by the ultrasound device, which provides guidance to the user, is a two dimensional slice of the patient's anatomy and the biopsy needle. The conventional methods and systems, however, suffer from the fact that even a small translation or rotation of the plane of the imaging device, or the bending of the medical device, leaves the medical device out of view.

Thus, there is a need in the field of medical imaging to create a new and useful system and method for displaying the trajectory of an instrument and the position of a body. The present invention provides such a new and useful method and system.

### BRIEF DESCRIPTION OF THE FIGURES

FIG. 1 is a schematic block diagram of a preferred system for displaying the trajectory of the instrument and the position of a body within a volume.

FIGS. 2-7 are various displays of the preferred system.

FIG. 8 is a flow chart depicting a preferred method for displaying the trajectory of the instrument and the position of a body within a volume.

### DESCRIPTION OF THE PREFERRED EMBODIMENTS

The following description of various preferred embodiments of the invention is not intended to limit the invention to these preferred embodiments, but rather to enable any person skilled in the art of medical imaging to make and use this invention.

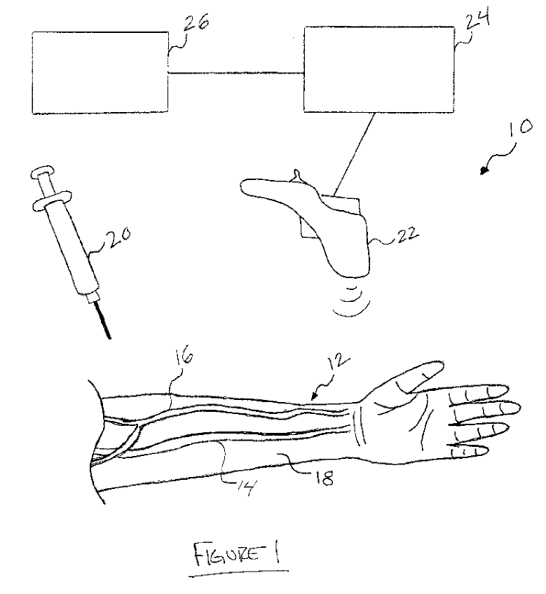

As shown in FIG. 1, a system 1o for displaying the trajectory of the instrument and the position of a body 14 within a volume 12 includes an instrument 20, an ultrasonic device 22 to propagate acoustic waves toward, and detect acoustic waves from, the volume 12, a processor 24 to create a representation of at least a portion of the volume 12 based on the detected acoustic waves and to calculate a trajectory of the instrument 20 relative to the body 14, and a display 26 to display the trajectory of the instrument 20 and the position of the body 14. The system 10 is preferably usable in the medical imaging arts, and in particular is well suited for use in the introduction of fluids into a vessel, such as a vein or artery, through a needle, syringe, or catheter. Alternatively, the preferred system 10 is usable in any context in which a use or operator must precisely maneuver an instrument into a body that is substantially hidden or obstructed from view.

The instrument 20 of the preferred system 10 functions to interact with the body 14. Preferably, the instrument 20 is a medical instrument (such as a syringe, a catheter, a fiber optic device, or a stent) that functions to penetrate the volume 12 and to transmit or collect fluids or other materials or to perform another medical function to the body 14. The body 14 is typically an artery or vein within a human or animal, but may also include other tissues, organs, and systems that are treated with minimal invasiveness, such as for example cardiac tissues and connective tissues. Alternatively, the preferred instrument 20 may be part of an automated system 10 that automatically guides the instrument 20 to the body 14 without an operator.

The ultrasonic device 22 of the preferred system 10 is adapted to propagate acoustic waves toward, and detect acoustic waves from, the volume 12. The preferred ultrasonic device 22 functions to create a three-dimensional representation of the volume 12, including information related to at least the position of the instrument 20 and the body 14. In particular, the ultrasonic device 22 is preferably of the type that emits ultrasonic waves that are reflected by one or more structures within the volume 12, such as the instrument 20, the body 14, any secondary bodies 16, and any other surrounding materials 18. The ultrasonic device 22 is preferably adapted to receive the reflected waves and provide data indicative of the frequency of the reflected waves. In a first alternative embodiment, the ultrasonic device is adapted to be at least temporarily fastenable to the instrument 20. Other suitable imaging devices such as MRI, CT and PET devices are usable as part of the system 10 in the generation of a three-dimensional representation of the volume 12.

The processor 24 of the preferred system 10, which is connected to the ultrasonic device 22, functions to generate a representation of at least a portion of the volume 12 based on the detected acoustic waves, and to calculate a trajectory of the instrument 20 relative to the body 14. The processor 24 creates the representation of at least a portion of the volume 12 through comparing those waves propagated by the ultrasonic device 22 to those detected by the ultrasonic device 22. The frequency of the detected waves is indicative of a structure, such as for example the body 14 or the instrument 20, and the processor 24 adapted to represent such structures based upon known values or value ranges of the detected frequency. In a variation, the processor 24 may be further adapted to segment the representation of the volume 12 into a least representations of the instrument 20 and the body 14. The processor 24 preferably segments the representation of the volume 12 in conjunction with calculating the trajectory of the instrument 20 relative to a position of the body 14. Segmentation of the representation of the volume 12 functions to clearly identify the instrument 20 and the body 14 by segmenting portions of a three-dimensional representation of the volume 12 into one or more two-dimensional segments that are displayable on the display 26. The processor 24 preferably allows for a rotation of the field of view that is independent of the orientation of the transducer.

The display 26 of the preferred system 10, which is connected to the processor 24, functions to display the trajectory of the instrument 20 and the position of the body 14. The preferred display 26 functions to aid an operator in the precise intersection of the instrument 20 and the body 14. The display 26 preferably includes a monitor, such as a CRT, LCD, or plasma screen, that is either a distinct element or integrated with the processor 24. The display 26 may alternatively include an audio component, such as for example a speaker or any other suitable device, to aid the operator in the precise intersection of the instrument 20 and the body 14.

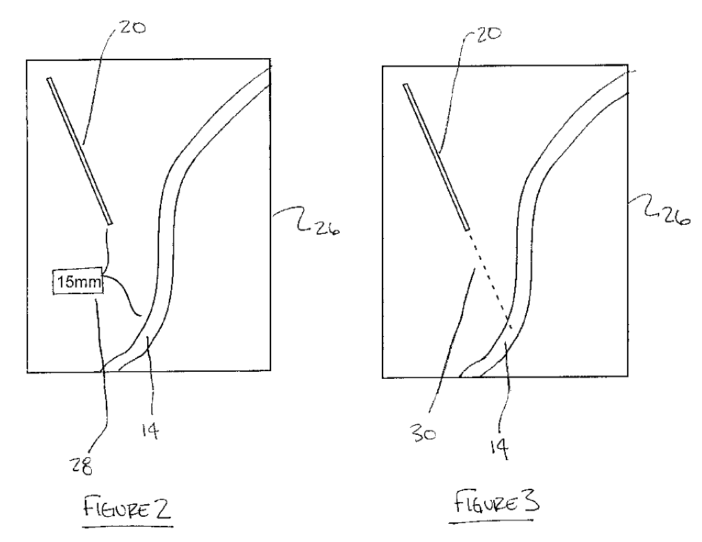

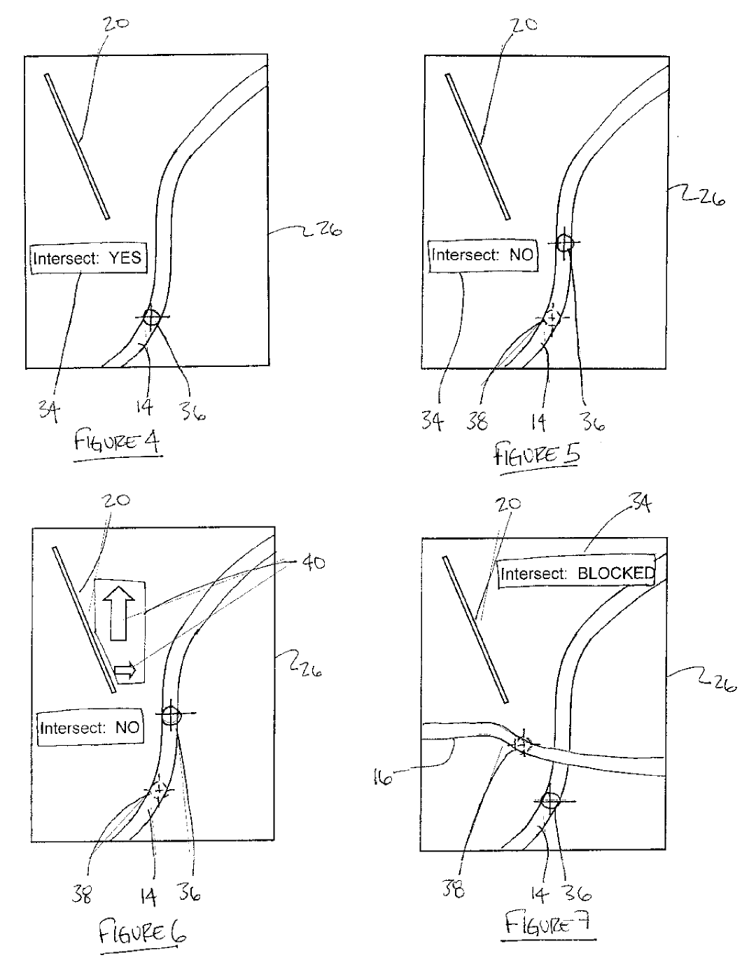

In a first alternative embodiment, the processor 24 and the display 26 calculate and display instance information based on the trajectory of the instrument 20 and the relative positions of the instrument 20 and the target. The instance information is preferably displayed continuously or substantially continuously during a medical procedure in which an instrument 20 is introduced into the volume 12 for the purpose of intersecting with a target defined on the body 14. Displaying the instance information functions to communicate to the operator of the instrument 20 readily processed instance information regarding the instrument 20 and the body 14. Preferably, the display 26 operates in conjunction with the processor 24, which is preferably adapted to calculate the instance information based on the trajectory of the instrument 20 and the relative positions of the instrument 20 and the target and communicate said calculation to the display 26. Examples of instance information calculable by the processor 24 and displayable by the display 26 are shown in FIGS. 2 through 6. As shown in FIG. 2, suitable instance information includes a distance 28 between the instrument 20 and the target on the body 14. As shown in FIG. 3, other suitable instance information includes a trajectory 30 of the instrument as it approaches the body 14 (shown with a dotted line or any other suitable indicator). As shown in FIGS. 4, other suitable instance information includes a prediction 34 of an intersection between the instrument 20 and a selected target 36. Preferably, the prediction may be conveyed through a window 34 on the display and through readily discernable language. Alternatively, as shown in FIG. 5, the instance information may include a non-selected target 38 that is distinct from the selected target 36 for communicating a failure to intersect in conjunction with the window 34. As shown in FIG. 6, other suitable instance information includes the presence of an intervening object, such as a secondary body 16 disposed between the instrument 20 and the target 36. Each of the foregoing examples of instance information may be displayed alone or in conjunction with one another by the display 26 of the system 10.

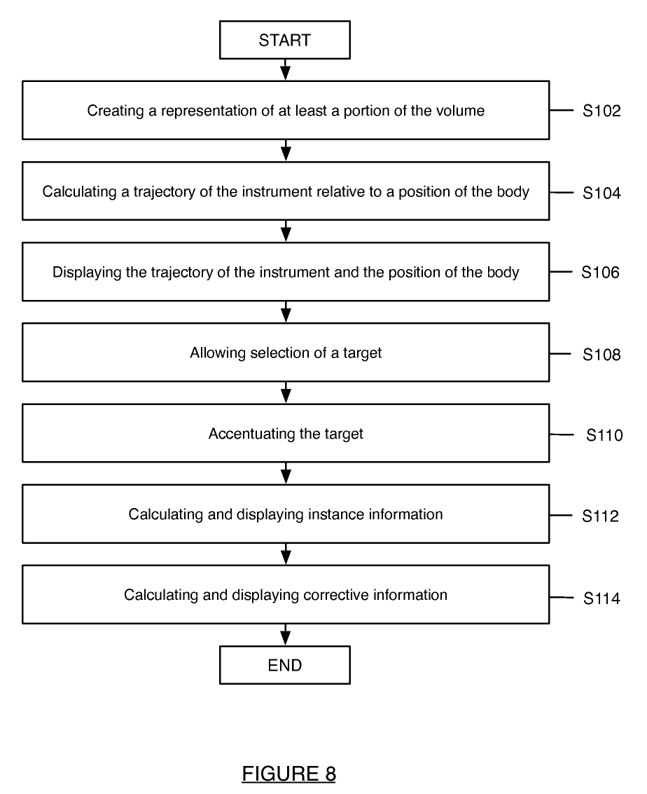

In a second alternative embodiment, the processor 24 and the display 26 calculate and display corrective information based upon the trajectory of the instrument 20 and the relative positions of the instrument 20 and the target. The corrective information is preferably displayed continuously or substantially continuously during a medical procedure in which an instrument 20 is introduced into the volume 12 for the purpose of intersecting with a target defined on the body 14. Preferably, the display 26 operates in conjunction with the processor 24, which is preferably adapted to calculate the corrective information based on the trajectory of the instrument 20 and the relative positions of the instrument 20 and the target and communicate said calculation to the display 26. The display 26 functions to communicate to the operator of the instrument 20 readily processed corrective information to aid in the introduction of the instrument 20 into the body 14. While the corrective information is preferably displayed visually by the display 26 of the system 10, one or more speakers or other suitable sound devices may communicate the corrective information in an aural format. As a variation, the corrective information may include actions to increase the possibility of an intersection between the instrument 20 and the target. Preferred actions to increase the possibility of an intersection between the instrument 20 and the target include displaying one or more arrows indicative of corrections to be made to the instrument trajectory in order to intersect the target, as shown in FIG. 7. Alternatively, the actions may include other visual or aural communications to the operator that indicate a more preferred trajectory for the instrument 20 in order to increase the possibility of an intersection with the target. Preferably, the actions are performed by the display 26 of the system 10, which is well suited for conveying visual corrective information. Alternatively, one or more speakers or other suitable sound devices may communicate the actions in an aural format.

As shown in FIG. 8, the method of the preferred embodiment includes seven steps for displaying the trajectory of the instrument 20 and the position of a body 14 within a volume 12. Step S102 includes creating a representation of at least a portion of the volume 12. Step S104 includes calculating a trajectory of the instrument 20 relative to a position of a body 14. Step S106 includes displaying the trajectory of the instrument 20 and the position of the body 14. Step S108 includes allowing a selection of a target. Step Silo includes accentuating the target. Step S112 includes calculating and displaying instance information based on the trajectory of the instrument 20 and the relative positions of the instrument 20 and the target. Step S114 includes calculating and displaying corrective information based on the trajectory of the instrument 20 and the relative positions of the instrument 20 and the target. The preferred method is best performed by the system 10 in a medical imaging context, although other suitable uses for the method include any context in which a use or operator must precisely maneuver an instrument into a body that is hidden, or substantially obstructed, from view.

Step S102 includes creating a representation of at least a portion of the volume 12. Step S102 functions to assemble and process data regarding the volume 12 in a manner that can be readily displayed by the display 26. Step S102 is preferably performed continuously or substantially continuously during a medical procedure in which the instrument 20 is introduced into a volume 12 for the purpose of intersecting with a target defined on a body 14. Step S102 is preferably performed by the system 10, including the ultrasonic device 22, the processor 24, and the display 26. Alternatively, step S102 can be readily performed by alternative imaging devices, such as MRI, CT, and PET devices, that are adapted for creating a representation of at least a portion of a volume 12 in response to known electrical, electromagnetic, chemical, or radiological properties of the volume 12. In the performance of step S102, the processor 24 preferably receives and processes volumetric and time information about the volume 12, and the display 28 is preferably displays this information.

Step S102 preferably includes the substep of propagating acoustic waves toward, and detecting acoustic waves from, the volume 12, and creating the representation based on the detected acoustic waves. The substep of step S102 is preferably performed by the ultrasonic device 22 and processor 24 of the system 10. As is known in the art, an ultrasonic device 22 is well suited for discriminating between structures having different acoustic properties, and in particular structures within which a fluid is flowing. Owing to the Doppler effect, the ultrasonic device 22 will detect acoustic waves that are distinct for vessels in which a fluid is flowing, as the motion of the fluid causes the frequency of the acoustic waves to be red-shifted by a known amount. As such, a preferred ultrasonic device 22 provides three-dimensional data representing at least a portion of a volume 12, including any body 14 or instrument 20. In the performance of the substep to step S102, the processor 24 preferably receives and processes volumetric and time information about the volume 12, and the display 28 is preferably displays this information.

Step S104 includes calculating a trajectory of the instrument 20 relative to a position of the body 14. Step S104 is preferably performed continuously or substantially continuously during a medical procedure in which an instrument 20 is introduced into the volume 12 for the purpose of intersecting with a target defined on the body 14. Step S104 functions to inform an operator of the instrument 20 of the projected path of the instrument 22 as it approaches the body 14. Step S104 is preferably performed by the system 10, including the ultrasonic device 22, the processor 24 and the display 26. The processor 24 preferably receives time information associated with the instrument 20, from which the processor 24 can calculate a trajectory of the instrument 20 relative to the position of the body 14.

Step S104 preferably includes a first substep of segmenting the representation of the volume into at least representations of the instrument 20 and the body 14. The first substep of step S104 is preferably performed in conjunction with calculating the trajectory of the instrument 20 relative to a position of the body 14. The first alternative of step S104 functions to clearly identify the instrument 20 and the body 14 through the segmentation process, which is preferably performed by the processor 24 of the system 10.

Step S104 preferably includes a second substep of further segmenting the representation of the body 14 into representations of multiple sections of the body 14. The second substep of step S104 is preferably performed in conjunction with calculating the trajectory of the instrument 20 relative to a position of the body 14. The second substep S104 functions to clearly identify portions the body 14, including for example a target, through the segmentation process, which is preferably performed by the processor 24 of the system 10.

Step S104 preferably includes the third substep of further segmenting the representation of the body 14 into a representation of fluid flow. The third substep of step S104 is preferably performed in conjunction with calculating the trajectory of the instrument 20 relative to a position of the body 14. The third alternative of step S104, which functions to clearly identify the position of the body 14, is preferably performed by the ultrasonic device 22 (using the Doppler effect described above) in conjunction with the processor 24 of the system 10, but may alternatively be performed using motion tracking correlation or any other suitable method.

Step S104 preferably includes the fourth substep of comparing the time information associated with the instrument 20. The fourth substep of step S104 is preferably performed in conjunction with calculating the trajectory of the instrument 20 relative to a position of the body 14. The fourth alternative of step S104 functions to identify the position of the instrument 20 at two or more distinct times during the medical procedure. The fourth substep of step S104 is preferably performed by the ultrasonic device 22 in conjunction with the processor 24 of the system 10. The processor 24 preferably receives time information associated with the instrument 20, from which the processor 24 can calculate a trajectory of the instrument 20 relative to the position of the body 14.

Step S106 includes displaying the trajectory of the instrument 20 and the position of the body 14. Step S106 is preferably performed continuously or substantially continuously during a medical procedure in which an instrument 20 is introduced into the volume 12 for the purpose of intersecting with a target defined on the body 14. Step S106 functions to inform an operator of the instrument 20 as to the trajectory of the instrument 20 and the position of the body 14 in order to efficiently and accurately perform a medical procedure. Step S106 may include the ability to rotate the field of view independent of the orientation of the transducer. Step S106 is preferably performed by the display 26 of the system 10, which in alternative embodiments may be a discrete component of the system 10 or integrated into the instrument 20.

Step S106 preferably includes a first substep of accentuating at least one of the multiple sections of the body 14. The first substep of step S106 is preferably performed in conjunction with displaying the trajectory of the instrument 20 and the position of the body 14. The first alternative of step S106 functions to clearly display portions the body 14, including for example a target, through the accentuation process, which is preferably performed by the processor 24 in conjunction with the display 26 of the system 10.

Step S106 preferably includes a second substep of accentuating the fluid flow. The second substep of step S106 is preferably performed in conjunction displaying the trajectory of the instrument 20 and the position of the body 14. The second alternative of step S106 functions to clearly identify the position of the body 14 through accentuation of the fluid flow therein. The fluid flow is preferably detected by the ultrasonic device 22 through the Doppler effect. Accentuation of the fluid flow is preferably performed by the processor 24 in conjunction with the display 26 of the system 10.

Step S108 includes allowing a selection of a target. Step S108 is preferably performed by an operator or technician associated with a medical procedure prior to the introduction of the instrument 20 into the volume 12 for the purpose of intersecting with a target defined on the body 14. Step S108 may, however, be performed by a machine. Preferably, step S108 is performed by the system 10, including the processor 24 and the display 26, which are readily adapted to allow a user to select a target on the body 14, the position of which is calculated by the processor 24 and displayed by the display 26. Alternatively, step S108 may be performed through the introduction of a known radiological, electromagnetic, chemical, or acoustic element into a target on the body 14 that renders that element identifiable by the system 10.

Step S110 includes accentuating the target, as selected in step S108 of the preferred method. Step S110 is preferably performed continuously or substantially continuously during a medical procedure in which an instrument 20 is introduced into the volume 12 for the purpose of intersecting with a target defined on the body 14. Step Silo functions to provide an operator of the instrument 20 with an easily identifiable rendering of the target on the body 14, such as for example a cross-hair displayed on the display 26 on the body 14. Alternatively, step S110 can be accomplished through the introduction of a known radiological, electromagnetic, chemical, or acoustic element into a target on the body 14 that accentuates the target for the operator of the instrument 20.

Step S112 includes calculating and displaying instance information based on the trajectory of the instrument 20 and the relative positions of the instrument 20 and the target. Step S112 is preferably performed continuously or substantially continuously during a medical procedure in which an instrument 20 is introduced into the volume 12 for the purpose of intersecting with a target defined on the body 14. Step S112 functions to communicate to the operator of the instrument 20 readily processed instance information regarding the instrument 20 and the body 14. In a first variation, instance information includes the distance between the instrument 20 and the target on the body 14. In a second variation, the instance information includes the prediction of an intersection between the instrument 20 and the target. In a third variation, the instance information includes the presence of an intervening object between the instrument 20 and the target. Each of the foregoing examples of instance information may be displayed alone or in conjunction with one another by the display 26 of the system 10.

Step S114 includes calculating and displaying corrective information based on the trajectory of the instrument 20 and the relative positions of the instrument 20 and the target. Step S114 is preferably performed continuously or substantially continuously during a medical procedure in which an instrument 20 is introduced into the volume 12 for the purpose of intersecting with a target defined on the body 14. Step S114 functions to communicate to the operator of the instrument 20 readily processed corrective information to aid in the introduction of the instrument 20 into the body 14. Step S114 is preferably performed by the display 26 of the system 10, which is well suited for conveying visual corrective information. Alternatively, one or more speakers may be coupled to the processor 24 or display 26 in order to communicate the corrective information in an aural format. The corrective information includes actions to increase the possibility of an intersection between the instrument 20 and the target. Preferred actions to increase the possibility of an intersection between the instrument 20 and the target include displaying one or more arrows indicative of corrections to be made to the instrument trajectory in order to intersect the target. Alternatively, the actions may include other visual or aural communications to the operator that indicate a more preferred trajectory for the instrument 20 in order to increase the possibility of an intersection with the target. Preferably, the actions are performed by the display 26 of the system 10, which is well suited for conveying visual corrective information. Alternatively, one or more speakers may be coupled to the processor 24 or display 26 in order to communicate the actions in an aural format.

As a person skilled in the art of medical imaging will recognize from the previous detailed description and from the figures and claims, modifications and changes can be made to the preferred embodiments of the invention without departing from the scope of this invention defined in the following claims.

I claim:

1. A method of displaying the trajectory of an instrument and the position of a body within a volume, comprising the steps of:
creating a representation of at least a portion of the volume;
calculating a trajectory of the instrument relative to a position of the body; and
displaying the trajectory of the instrument and the position of the body.

2. The method of claim 1 wherein the creating step includes propagating acoustic waves toward, and detecting acoustic waves from, the volume, and creating the representation based on the detected acoustic waves.

3. The method of claim 1 wherein the calculating step includes segmenting the representation of the volume into at least representations of the instrument and the body.

4. The method of claim 3 wherein the creating step includes collecting volumetric and time information about the volume.

5. The method of claim 4 wherein the calculating step includes comparing the time information associated with the instrument.

6. The method of claim 3 wherein the segmenting substep includes further segmenting the representation of the body into representations of multiple sections of the body.

7. The method of claim 6 wherein the displaying step includes accentuating at least one of the multiple sections of the body.

8. The method of claim 3 wherein the segmenting substep includes further segmenting the representation of the body into a representation of fluid flow.

9. The method of claim 8 wherein the displaying step includes accentuating the fluid flow.

10. The method of claim 1 further comprising the step of allowing selection of a target; and wherein the displaying step further includes accentuating the target.

11. The method of claim 10 further comprising the step of calculating and displaying instance information based on the trajectory of the instrument and the relative positions of the instrument and the target.

12. The method of claim 11 wherein the instance information includes the distance between the instrument and the target.

13. The method of claim 11 wherein the instance information includes the prediction of an intersection between the instrument and the target.

14. The method of claim 11 wherein the instance information includes the presence of an intervening object between the instrument and the target.

15. The method of claim 10 further comprising the step of calculating and displaying corrective information based on the trajectory of the instrument and the relative positions of the instrument and the target.

16. The method of claim 15 wherein the corrective information includes actions to increase the possibility of an intersection between the instrument and the target.

17. A system for displaying the trajectory of the instrument and the position of a body within a volume, comprising:
an instrument;
an ultrasonic device adapted to propagate acoustic waves toward, and detect acoustic waves from, the volume;
a processor coupled to the ultrasonic device, adapted to create a representation of at least a portion of the volume based on the detected acoustic waves, and adapted to calculate a trajectory of the instrument relative to the body; and
a display adapted to display the trajectory of the instrument and the position of the body.

18. The system of claim 17 wherein the instrument is a medical instrument adapted to interact with the body.

19. The system of claim 18 wherein the medical instrument and the ultrasonic device are adapted to be at least temporarily fastenable to each other.

20. The system of claim 17 wherein the processor is further adapted to segment the representation of the volume into at least representations of the instrument and the body.
